(19) United States
(12) Patent Application Publication
(10) Pub. No.: US 2005/0130231 A1
(43) Pub. Date: Jun. 16, 2005

(54) Method for analyzing effects of medical agents

(76) Inventor: Yuping Deng, Yorktown, VA (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(21) Appl. No.: 10/499,320

(22) PCT Filed: Dec. 20, 2002
(86) PCT No.: PCT/US02/40797

(30) Foreign Application Priority Data
Dec. 21, 2001 (US) 60342432

(51) Int. Cl. G01N 33/53; G01N 33/567; A61K 35/78

(52) U.S. Cl. 435/7.2; 424/728

(57) ABSTRACT

A method for analyzing the effects of medicinal agents, and more particularly, an ex vivo method of analyzing the effects of medicinal agents on the human immune system is provided.

### BACKGROUND

Herbal medicines, such as ginsengs, have been considered as tonics and studies show that ginseng can improve the immune functions in humans. Extracts from ginsengs, including red ginseng and Panax ginseng (also called American ginseng) are reported to have anti-tumor effects in animal models and in humans. In addition, clinical trials show that ginseng extracts have prophylaxis effects on respiratory infections in older adults. The mechanism of how ginsengs may augment the immune system to combat respiratory infection is currently not known.

By better understanding how herbal medicines or the like work, and by learning how to screen these herbal medicines for health value, bacterial and viral infections such as influenza may be more quickly abated. Influenza infection is a serious public health problem. Influenza can be catastrophic for people in advanced age who are known to have impaired immune function. A herbal remedy that may boost the immune function to prevent influenza infection and to help recovery from influenza can provide much needed help for people.

The immune response to viral infection can be divided into two phases: the initial innate immune response and the subsequent adapted immune response. The innate immune response, acting on the frontier of combating viral infection, involves the activation of macrophages (also called monocytes in humans) and natural killer (NK) cells to secrete cytokines and chemokines. The cytokines/chemokines secreted by these cells can have a direct antiviral function, and also function to activate the adapted immune response. Adapted immune response is antigen-specific and hence, more sophisticated, and can be further divided into humoral (antibodies) and cell mediated immune responses. The major players in cell mediated immune response may include type 1 CD4 positive T helper cells (Th1) and CD8 positive cytotoxic T lymphocytes (CTL). Th1, in part, helps the proliferation of CTL, and CTL can kill viral infected cells directly via recognizing viral antigens.

### SUMMARY

A method for analyzing the effects of medicinal agents, and more particularly, an ex vivo method of analyzing the effects of medicinal agents on the human immune system is provided.

How medicinal agents effect cells and/or stimulate the immune system is largely unknown. In an attempt to delineate the potential benefit of medicinal agents, a method of analyzing the effects of a medicinal agent is provided herein. The method is accomplished by contacting the medicinal agent with cells and identifying cellular response to the medicinal agent. Contacting may be accomplished by co-incubating the medicinal agent with the cells or by other methods known in the art. The cellular response may be identified by probes for phenotype markers and/or chemical markers. Phenotype markers may be used to determine the type of cell affected by the medicinal agent, while the chemical markers may identify molecules that are produced by cells. Both the phenotype markers and chemical markers may be varied to identify a wide range of cells and/or molecules.

In part, a method for determining the effect of a medicinal agent on the immune response is provided herein. This is accomplished by contacting the medicinal agent with immuno-responsive cells to form an agent-cell mixture and providing at least one probe to the agent-cell mixture to assay for cellular activation. As used herein, “immuno-responsive cells” refers to any cell type that is activated directly or indirectly by a cellular bound or unbound antigen. The probes may include antibodies to cell-surface antigens, secreted antigens, and/or intra-cellular antigens. Examples of cell-surface antigens may include surface proteins or cell membrane proteins of immuno-responsive cells, or any other type of cell-surface antigen known in the art to identify cell type. Examples of intra-cellular and/or secreted antigens/molecules may include polypeptides such as chemokines and/or cytokines.

A method for determining cellular reaction to a medicinal agent by contacting the medicinal agent with immuno-responsive cells to form an agent-cell mixture, and assaying for the presence of at least two phenotype markers in the agent-cell mixture is also provided herein. Each phenotype marker is employed to identify a particular type of activated immuno-responsive cell. Examples of types of immuno-responsive cells includes T cells, B cells, natural killer cells, and monocytes. Additionally, at least one probe for a chemical marker may be contacted with the agent-cell mixture. Each chemical marker probe commonly identifies a particular protein, or class of proteins produced by immuno-responsive cells. Examples of proteins produced by immuno-responsive cells include cytokines and chemokines, although other cellular proteins may also be identified. Such proteins may be produced by a single cell type or may be produced by more than one type of cell.

The agent-cell mixture may be screened at various time intervals to identify the effects of the medicinal agent by analyzing the phenotype markers and/or chemical markers. Moreover, bacterial or viral agents may be added to the agent-cell mixture prior to screening the mixture for the presence of activated immuno-responsive cells. Screenings may take place at time intervals chosen for the particular test, including by the minute, hourly, and/or daily screens, depending on the desired parameters and markers used.

Probes for other types of markers may also be added to the agent-cell mixture including probes for activation markers. Activation markers are produced by cells that become activated and commonly either suppress or enhance transcription of particular peptides in the presence of different substances. Illustrative examples of activation markers include CD69 (corresponding to T-cells and monocytes) and MHC Class II (otherwise known as HLA-DR, which corresponds to B-cells). Activation markers may also help identify the effects of medicinal agents on a variety of cell types.

A method of detecting cellular response by incubating peripheral blood mononuclear cells in the presence of a plant-based material and contacting the incubated peripheral blood mononuclear cells with a set of probes to form a complimentary probe-cell complex is also provided herein. Each probe is capable of complexing with a specific phenotype marker for an immuno-responsive cell type. The probe-cell complexes may then be detected and analyzed. Probes may also be used for chemical and/or activation markers, and the probe-marker complexes may also be detected.

### BRIEF DESCRIPTION OF THE DRAWINGS

FIG. 1. Dose-dependent activation of Panax ginseng extract on CD14+ monocytes.

FIG. 2A. Specific activation of NK cells by Panax ginseng extract in the presence of influenza viruses.

FIG. 2B. Dose-dependent activation of NK cells by ginseng extract.

FIG. 3. Comparison of different Panax ginseng extract in activating NK cells to secrete IFN-g.

FIG. 4. Effect of Panax ginseng extracts on the proliferation of Th1 CD4 positive T cells and CD8 positive CTL.

FIG. 5. Effect of Panax ginseng extract (CVT 2001 009) on the growth of NK cells in CLT cultures stimulated by killed influenza viruses.

### DETAILED DESCRIPTION

A method for analyzing the effects of medicinal agents has been developed. Medicinal agents may include both naturally-derived and synthetically manufactured agents. Examples of a naturally-derived medicinal agents include plant based materials, such as herbs. Plant based materials may include, without limitation, leaves, roots, bark, sap, berries, and/or extracts or combinations thereof. Likewise, the plant based material may be ground, dried, fractionated, seeped, or left whole.

In one embodiment, the medicinal agents are contacted with peripheral blood mononuclear cells (PBMC), which is a mixture of cell types including immuno-responsive cells such as Th1, CTL, NK, B-cells and monocytes (otherwise known as macrophages). Each of these immuno-responsive cell types has at least one phenotype marker such as a surface antigen capable of being detected. For example, CD4, CD8, CD56, CD19, and CD 14 are phenotype markers for Th1, CTL, NK, B-cells and monocytes, respectively. Th1 and CTL cells also have antigen CD3. NK cells have additional antigen CD16. Each surface antigen (phenotype marker) may be identified by a probe, which is typically an antibody to these surface antigens but may be any other probe that can be used to identify the surface antigens. The probes can be labeled with a radioactive, fluorescent, or colored tag, although other types of labels known in the art may be used.

Immuno-responsive cells commonly produce different chemical markers when activated, e.g., peptides and/or other factors. Many of these chemical markers are known as cytokines and chemokines. The cytokines include the interleukins, such as IL-2, IL-4, IL-6, IL-10, and IL-12. Other cytokines include interferon-gamma (ifn-γ) and tumor necrosis factor-alpha (tnf-α), although other cytokines may also be produced by immuno-responsive cells. Examples of suitable chemokines (low molecular weight polypeptides that chemotactically attract different leukocytes) which may be assayed for include macrophage chemotatic and activating factor (MCAF), macrophage inflammatory protein-1a (MIP-1a), macrophage inflammatory protein-1b (MIP-1b), RANTES, and interleukin 8 (IL-8). These chemical markers may also be identified using probes, which are often antibodies to each of these peptides and/or factors but may be any probe that identifies a specific secretory or intra-cellular peptide or factor produced from an immuno-responsive cell.

For detecting cellular response, PBMC may be incubated in the presence of a medicinal agent, such as a herbal medicinal agent. Incubated PBMC may then be contacted with a set of probes for at least two phenotype markers for specific immuno-responsive cell types (e.g. Th1 cell and B-cell). Each probe specifically identifies a particular phenotype marker. When the phenotype marker is present within the incubated cells and is contacted by its specific probe, a probe-marker complex is formed. The probe-marker complexes can then be detected and analyzed by methods and assays known by those skilled in the art.

Additional components may be added to the incubated PBMC to provide more information about the effects of the medicinal agent. For example, probes for specific chemical markers like cytokines and chemokines may be contacted with the incubated PBMC to form additional probe-marker complexes that may be detected and analyzed. Infectious agents, such as bacterial or viral agents, may also be incubated with the PBMC, in combination with probes for phenotype markers and/or chemical markers. Finally, other types of probes may be used to contact the incubated PBMC including probes for activation markers or other markers known by those skilled in the art. Other types of agents known in the art may also be incubated with PBMC and medicinal agent to determine whether any benefit or detriment is achieved.

The following examples illustrate the method of analyzing the effect of medicinal agents. These examples illustrate but do not limit the scope of the invention that has been set forth herein. However, before discussing each example, the experimental background will be discussed to provide a sufficient understanding of the methods employed to reach the results of each example. Of course, these methods may be varied or changed as known in the art to achieve the same or similar results, or the methods may be improved upon or altered as technology improves.

Panax ginseng extracts: The medicinal agent chosen for the following examples was Panax ginseng. Panax ginseng extracts were provided by CV Technologies (CVT), Edmonton, Canada. There were three different lots of Panax ginsehg extracts, CVT 2001 008, CVT 2001 009, CVT 2001 010, and two different lots of control extracts CVT HT 1001-005 and CVT HT 1001-009. All extracts were provided in powder form and were dissolved in PBS buffer. Extracts were then diluted into final concentrations in tissue culture medium, RPMI, (Gibco, Md.) with 10% FBS (Summit Biotech, CO).

Purification of peripheral blood mononuclear cells: Heparinized PBMC (20 to 30 ml each blood draw) from healthy individuals was spun at 1200 RPM for 10 minutes to isolate buffy coat layers. Buffy coat layers were collected and carefully laid on top of a ficoll gradient (Histopaque, Sigma, Mo.). Cells were then centrifuged for 30 min. at 1700 RPM. PBMC were collected from the interface, spun down, and washed twice with RPMI medium before used for experiments.

Analysis of activation of monocytes by Panax ginsengs: 5×10⁵ PBMC in 200 ul of AIM-V medium (Gibco, Md.) were incubated with different doses of Panax ginseng extracts overnight (16 hours). Cells were spun down, stained with surface antigen antibodies CD14-APC, CD69-FITC and CD86-PE. Stained cells were subjected to flow cytometry analysis using a FACSCalibur (Becton Dickinson, Calif.) and data were analyzed by CellQuest software (Becton Dickinson, Calif.). CD14 positive cells were gated and their expression of activation markers CD69 and CD86 were analyzed using appropriate antibodies.

Analysis of activation of dendritic cells (DC) by ginseng:

Dendritic cells were generated by methods known to those skilled in the art, with the following brief example provided for illustration. 1.5×10⁷ freshly isolated PBMC in 3 ml of AIM-V were placed into wells of a 6-well plate and incubated for 3 hours at 37° C. Non-adherent cells were taken out. Adherent cells were washed twice gently, and were cultured in 5 ml AIM-V with the presence of GM-CSC (800 Unite/ml) and IL-4 (1000 Unite/ml) (BioSource, CA). After 7 days of incubation, cultures usually contained more than 50% dendritic cells, as determined by the cultures' dendrite-rich morphology and high expression of MHC and co-stimulatory molecules. To analyze the effect of ginseng in dendritic cells, cells from dendritic cells cultures were harvested on day 7, counted and incubated with different doses of Panax ginseng extracts overnight. Cells were then stained with CD86, 83, and MHC II (also called HLA-DR) antibodies.

FastImmune assay: 1×10⁶ PBMC were co-incubated with different doses of Panax ginseng extracts and controls. A mixture of live influenza viruses that contain equal amounts (10 HA Unite/ml) of influenza A/H3N2/Calvadonia, A/H1N1/Panama and B/Yamanashi was added to PBMC as needed. The allantoic fluid was used as the control antigen for the live influenza viruses. PBMC were activated with or without influenza for 3 hours and Brefeldin A (BFA, 5 ug/ml, Sigma, Mo.) was added to the cells. PBMC were incubated for another 15 hours. The PBMC were then fixed (1% Paraformaldehyde, Sigma, Mo.), permeablilized (permeablilyzation buffer, Becton Dickinson, Calif.), and stained for the following conjugated antibodies: CD56-PE, CD4-APC, CD8-PerCP, and IFN-g-FITC. Stained PBMC were subjected to flow cytometry analysis using a FACSCalibur cytometer and CellQuest software. Lymphocytes were gated from scattergraph for subsequent analyses. NK cells (CD56 positive) that were also positive for IFN-g were defined as activated NK cells.

CTL cultures: 1.5×10⁶ PBMC in 1.5 ml of complete medium (RPMI plus 10% FBS) were placed into wells of 24-well plate with different doses of ginseng extract. PBMC were stimulated with killed influenza viruses (from 1:1000 diluted influenza vaccine) of influenza A Calvadonia, A Panama and B Yamanashi. Cultures were supplemented with a low dose of IL-2 (20 IU/ml) and IL-7 (20 ng/ml) every 48 hours. After 7 to 9 days of culture, cells were harvested, counted and analyzed for the frequency of influenza-specific T cells.

Quantification of influenza-specific CD4 positive or CD8 positive T cells from CTL cultures: On the day the CTL cultures were harvested for analysis, peripheral blood was also obtained from the original donors whose PBMC were used for setting up the CTL cultures. Freshly isolated PBMC were infected by influenza viruses. These freshly isolated PBMC were to provide autologous antigen presenting cells. Infected PBMC were mixed with the cells harvested from the CTL culture at a ratio of 1:10 (PBMC:CTL) respectively. As a control, uninfected PBMC were used to mix with cells from CTL cultures. Mixtures of cells were incubated for 3 hours before BFA was added. Influenza-specific T cells were quantified by FastImmue as similar to that described above. The frequency of influenza-specific T cells was defined as CD8 positive or CD4 positive T cells that were also IFN-g positive upon stimulation by influenza-infected cells.

### EXAMPLE 1

### Effect of Ginseng on Antigen Presenting Cells such as Monocytes and Dendritic Cells

Antigen presenting cells, which include monocytes or macrophages and dendritic cells, are important cells in mediating T cell response by processing and presenting viral antigens to T cells in the event of a viral infection. Activated antigen presenting cells are known to be more effective antigen presenting cells because of their higher expressing of MHC and co-stimulatory molecules. In addition, activated antigen presenting cells can secrete cytokines such as IL-12 and IL-10 to recruit T cells and to stimulate T cell to proliferate.

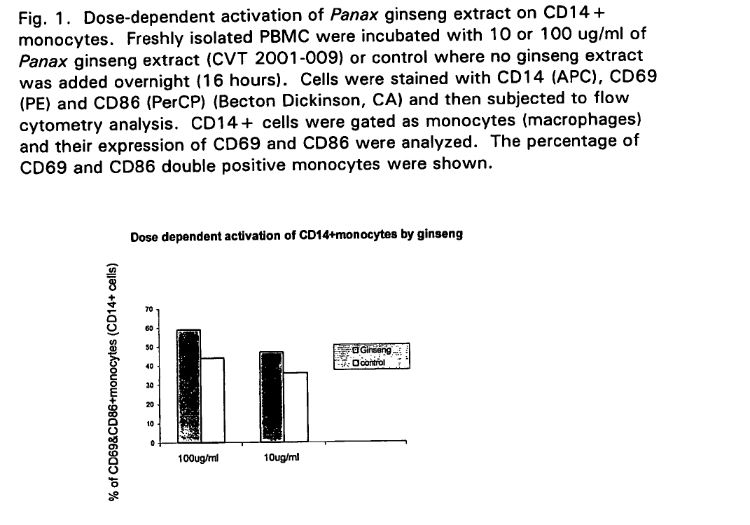

The following two experiments were conducted to determine the effects of ginseng on antigen presenting cells. First, PBMC was incubated with different doses of Panax ginseng extract over night (16 hours) to determine if monocytes (CD14 positive cells) could be activated by Panax ginseng. Activation marker CD69 and CD86 double positive monocytes were used as an indicator of activated monocytes. This first experiment resulted in an increase of activated monocytes in the presence of Panax ginseng extract, and the activation of monocytes was dose-dependent (FIG. 1).

The second experiment was to determine the effect of ginseng on in vitro generated dendritic cells, the professional antigen presenting cells. Dendritic cells were generated from adherent PBMC in the presence of GM-CSF and IL-4 for 7 days. Dendritic cells were then harvested and incubated with different doses of ginseng extract overnight, and the activation effect was analyzed by the potential increased expression of MHC II, CD86 and CD83. CD86 is a co-stimulating molecules (B7.2) and CD83 is a marker for mature dendritic cells. Activated dendritic cells are known to have increased expression of CD83, CD86 and MHC II. Results showed that there was an increase in CD83 (data not shown) after dendritic cells were co-incubated with ginseng extract overnight.

### EXAMPLE 2

### Effect of Panax Ginseng Extracts on Stimulating NK Cells in Response to Influenza Infection

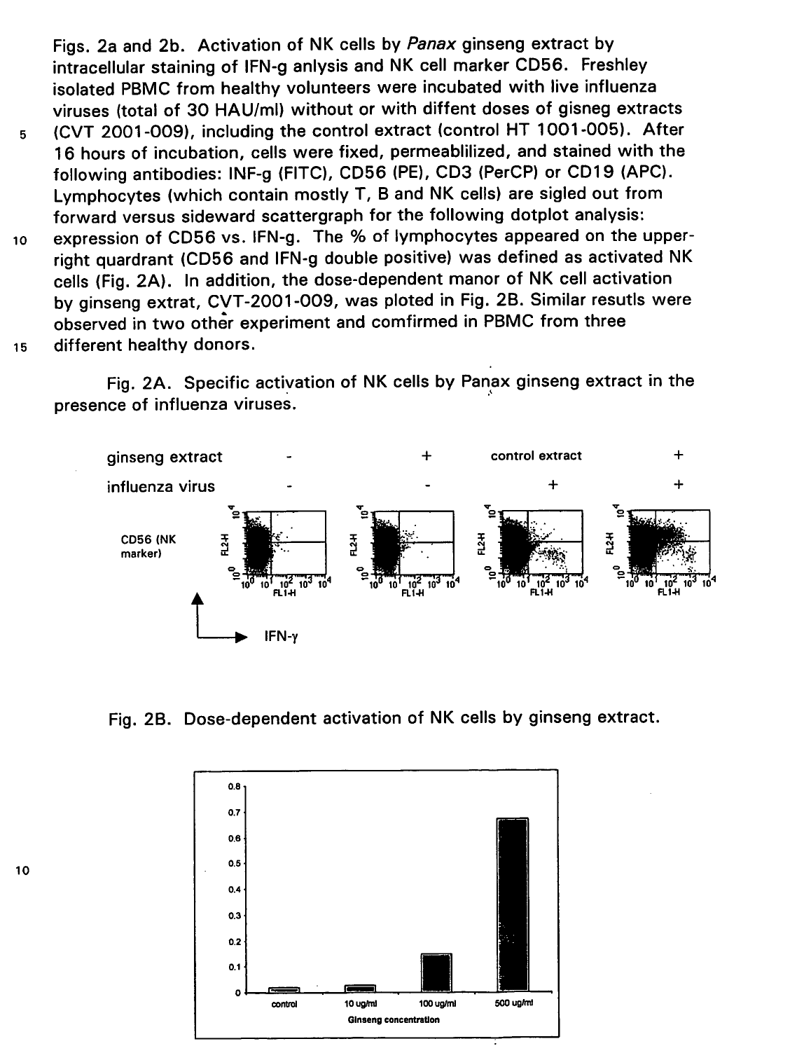

NK cells play an important role in the initial phase of a viral infection because NK cells are able to kill viral infected cells directly. Also, activated NK cells can secrete cytokines such as IFN-g and IL-2. IFN-g has both antiviral effect directly as well as the ability to provide help for the proliferation of CTL. Experiments were conducted to determine whether ginseng stimulated NK cells, which resulted in the finding that ginseng extract stimulated NK cells when PBMC were incubated overnight with live influenza viruses (FIG. 2A). The stimulation of ginseng was demonstrated by the secretion of IFN-g by NK cells. The activation of NK cells was ginseng extract dose-dependent (FIG. 2B). In addition, the activation of NK cells by ginseng was NK cell-specific because ginseng did not activate CD3 positive T cells (which screened for both Th1 and CTL cells) or NKT cells (CD3 and CD56 double positive cells). The stimulation of NK cell by ginseng was also found to be pathogen-dependent, i.e., ginseng stimulated NK cells to secrete IFN-g only in the presence of influenza viruses (FIG. 2A, second and fourth panel). Experiments were also conducted to test the two control ginseng extracts provide by CVT (CVT HT 1001-005 and CVT HT 1001-009). These control extracts were known to have no detectable immunological function. No NK cells were activated by these two control extracts even when influenza viruses were present in the culture (FIG. 2, third panel).

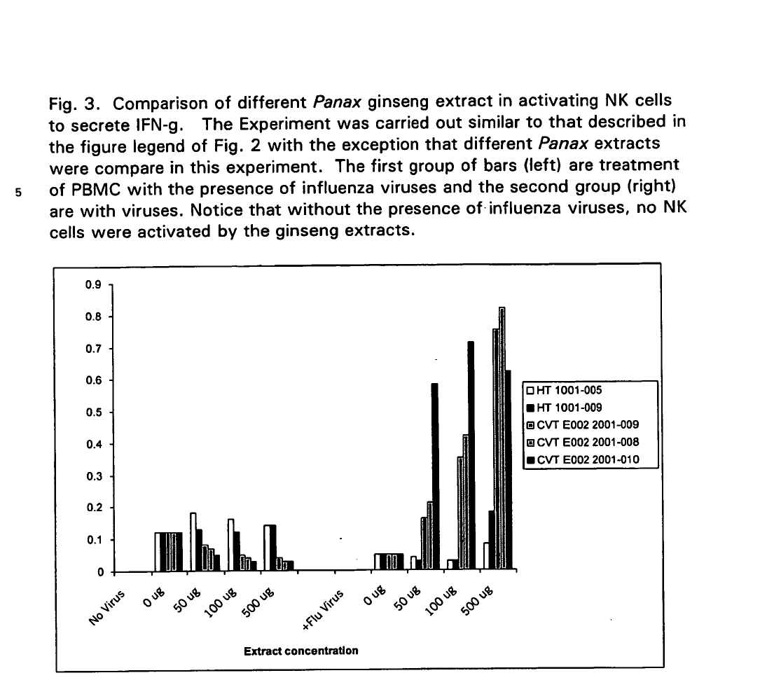

The experiments showed that ginsengs stimulate the immune system in response to influenza virus infection by stimulating/activating NK specifically. The activation of NK cells by ginsengs is influenza-specific, and ginseng extract dose-dependent. Out of all three ginseng extracts and two controls extracts tested, the ginseng extracts showed specific stimulation effect on NK cells to secrete IFN-g, with only very little variation in terms of the degree of activations by these three different extracts (see FIG. 3). Moreover, no stimulation of NK cells was seen by the two controls extracts, or when no ginseng was added.

### EXAMPLE 3

### Effect of Ginseng on the Growth of Influenza-Specific CD4 Positive Th1 Cells, CD8 Positive CTL Cells and NK Cells

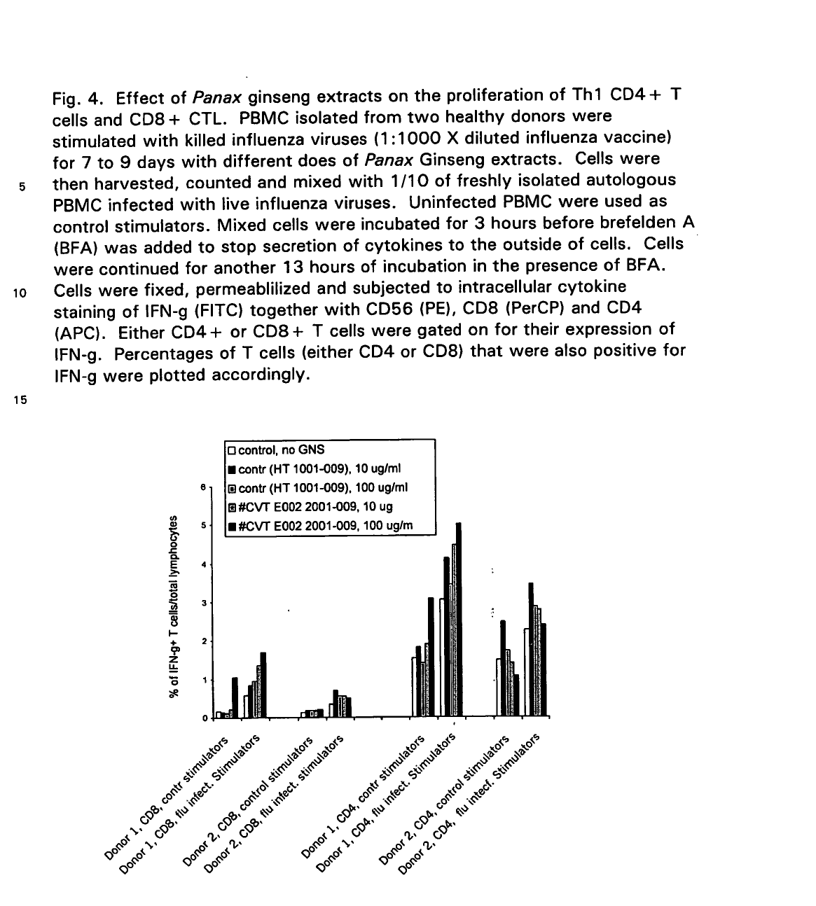

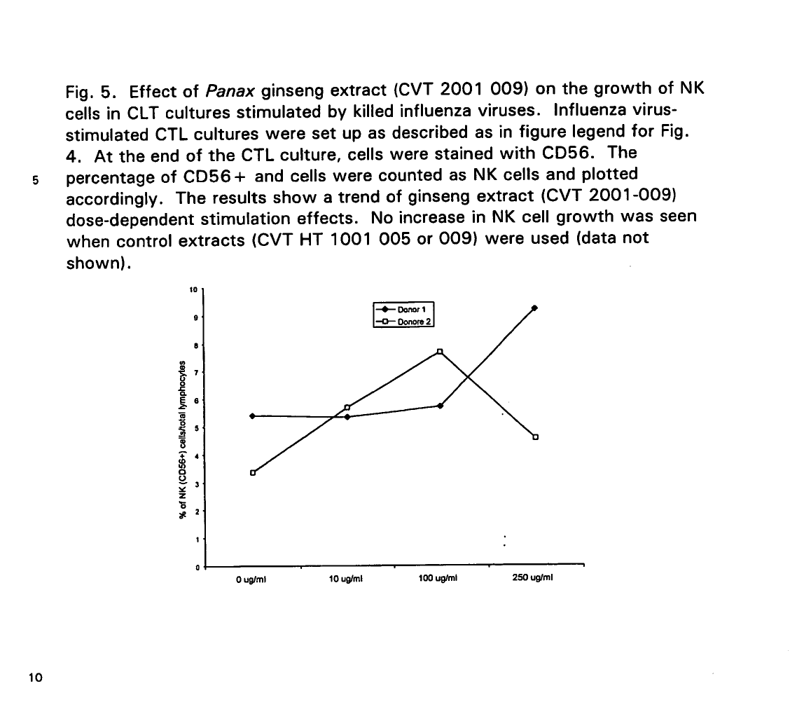

The experiments of Examples 1 and 2 above are ex vivo experiments that utilized short-term (overnight) culture of freshly isolated PBMC. Although ex vivo experiments are preferable in analyzing the cellular mechanism of how ginseng extracts work, ex vivo experiments could not be used for studying the long-term effect of ginseng extracts on the proliferation of CTL or other cells types. For that reason, CTL cultures of PBMC stimulated by diluted influenza vaccine (containing killed influenza viruses) were set-up in the presence of ginseng extracts. As controls, experiments were conducted using either the CVT control extracts or ginseng extract was added. After 7 to 9 days of co-incubation with vaccine, cells from CTL cultures were harvested, counted and subjected to FastImmune assay. IFN-g positive T cells were defined as antigen-specific T cells after reactivation by influenza infected antigen presenting cells (see material and method section). It was observed that in the CTL cultures from one donor, ginseng extract stimulated more influenza-specific T cells growth, particularly the CD8 positive CTL cells (donor 1, CD8+ cells, FIG. 4). It was also found that there were significant higher numbers of NK cells in the CTL culture when ginseng extract was added compared to the controls where no ginseng extract was added (FIG. 5). In addition, the results showed an increase of NK cells with ginseng in a ginseng extract dose-dependent manor. When control extracts (CVT HT 1001-005 or CVT HT 1001-009) were used, no increase of NK cell growth was seen (data not shown) in the CTL cultures. This result is consistent with the observation that Panax ginseng extracts stimulated NK cells (to secrete IFN-g) in the early phase of activation by influenza viruses (FIG. 2).

As can be seen by the above-identified examples, Panax ginseng extracts activate monocytes directly, and the presence of the extracts also augmented the NK cell activation in response to influenza infection. Panax ginseng extracts also help the proliferation of influenza-specific T cells, including Th1 CD4 positive T cells and CD8 positive CTL cells, as well as the proliferation of total NK cells.

It was also observed that influenza viruses induced the IFN-g secretion from influenza-specific memory T cells (low right quadrant in FIG. 2A, third penal). Panax ginseng extracts did not have augmentation effect on T cells in short-term culture in either influenza-specific or non-influenza-specific T cells. Although activation of NK cells can be seen by influenza viruses alone, even without the presence of ginseng extract, activation of NK cells by influenza-viruses requires a prolonged incubation (>16 hours) of PBMC with live influenza viruses. With ex vivo conditions where all cytokine secretions were stopped (by BFA) after 3 hours of co-incubation of PBMC and viruses, no activation of NK cells were seen unless the Panax ginseng extracts were added.

Finally, the results demonstrate the feasibility of an ex vivo analysis for studying the mechanistic of how medicinal agents may effect cells, including immuno-responsive cells. This provides a method for screening medicinal agents to determine whether they will be capable of stimulating the immune system. This ex vivo analysis can also be further utilized to analyze the immunological effects of medicinal agents, including herbal medicines, in response to a particular pathogen of interest.

1. A method of determining the effects of a medicinal agent on the immune response comprising:
contacting the medicinal agent with immuno-responsive cells to form an agent-cell mixture; and
assaying the agent-cell mixture for cellular activation with a probe.

2. The method of claim 1, wherein the probe used for assaying the agent-cell mixture for cellular activation includes a phenotype marker probe, chemical marker probe, activation marker probe, or a combination thereof.

3. The method of claim 1, wherein the probe used for assaying the agent-cell mixture for cellular activation includes an antibody to cell surface antigens, antibody to secreted antigens, antibody to intra-cellular antigens, or a combination thereof.

4. A method for determining cellular reaction to a medicinal agent comprising:
contacting the medicinal agent with immuno-responsive cells to form an agent-cell mixture; and
assaying the agent-cell mixture for the presence of at least two phenotype markers.

5. The method of claim 4, wherein the phenotype markers include CD4, CD8, CD56, CD19, CD14, CD3, CD16, or a combination thereof.

6. The method of claim 4, further comprising contacting the agent-cell mixture with a probe for a chemical marker and assaying the agent-cell mixture for the presence of the chemical marker.

7. A method of detecting cellular response comprising:
incubating peripheral blood mononuclear cells in the presence of a plant-based material; and
contacting the incubated peripheral blood mononuclear cells with a set of probes to form a probe-cell complex.

8. The method of claim 7, further comprising detecting the probe-cell complex.

9. The method of claim 7, further comprising contacting the incubated peripheral blood mononuclear cells with a probe for a chemical marker activation marker, or a combination thereof to form a probe-marker complex and detecting the probe-marker complex.

10. The method of claim 7, further comprising incubating the peripheral blood mononuclear cells in the presence of an infectious agent.

11. The methods of claims 1 and 4, wherein the medicinal agent includes Panax ginseng derived material.
